(19) United States
(12) Patent Application Publication
(10) Pub. No.: US 2024/0180773 A1
(43) Pub. Date: Jun. 6, 2024

(54) PREDICTION, SIMULATION, AND ANALYSIS TOOL FOR MECHANICAL DISRUPTION OF SOFT TISSUES

(71) Applicant: Rejuvatek Medical Inc., Sandy, UT (US)

(72) Inventors: Timothy N. Turner, West Jordan, UT (US); Jack H. Savage, Sandy, UT (US)

(21) Appl. No.: 18/530,631

(22) Filed: Dec. 6, 2023

Related U.S. Application Data
(60) Provisional application No. 63/430,575, filed on Dec. 6, 2022.

(51) Int. Cl. A61H 7/00; G16H 40/63

(52) U.S. Cl. CPC A61H 7/004; G16H 40/63; A61H 2201/1695; A61H 2201/5007; A61H 2201/5043; A61H 2201/5061

(57) ABSTRACT

Mechanical disruption of soft tissues is a treatment modality for many dermatological, cosmetic, and medical procedures that has defied quantification. When driven through soft tissue, sturdy mechanical implements are not affected by the tissue, which permits a useful separation of specified kinematics from unknown dynamics. Tissue disruption (breaking structural fibers and other bonds) is proportional to the mechanical energy density delivered to the tissues, which in turn is related to a novel quantity: the kinematic displacement density (“KDD”). A kinematic displacement tool, including software with auxiliary hardware, may be used for, among other purposes, calculating, displaying, and using KDD in various application categories, including but not limited to: simulation and analysis of mechanical tissue disruption for medical purposes; training disruption technicians; optimizing disruption implements; programming semi-automatic disruption devices; and measuring the actual mechanical energy density delivered to tissues.

### CROSS-REFERENCE TO RELATED APPLICATIONS

This application claims priority to U.S. Provisional Application No. 63/430,575 filed on 6 Dec. 2022, the contents of which are incorporated herein by reference.

### FIELD OF THE INVENTION

The present invention relates generally to systems, tools, devices, and methods for disruption of soft tissues and analysis of the disruption of soft tissues.

### BACKGROUND

Disrupting soft tissues of the skin (or other body organs) may be a significant treatment modality for many dermatological, cosmetic, and medical procedures. Tissue disruption implies alteration of the normal homeostasis or pathological imbalance that may be present. Tissue disruption can be imperceptible, when no histologically discernible structural damage is involved; it can be minor, when easily repairable damage is inflicted; or it can be major, when tissues are so destroyed that natural repair mechanisms must disintegrate the damage and totally rebuild the tissue, often imperfectly.

Imperceptible disruptions that induce no discernible structural damage to living tissue may include treatments like phototherapy, superficial peels, microdermabrasion, galvanic therapy, and botulinum toxin injection. Minor disruptions that inflict limited structural damage may include microneedling for collagen stimulation and scar revision, and tattoo pigment fragmentation with various directed energy sources (lasers, focused ultrasound, radio frequency waves, microwaves, etc.). Major disruptions that destroy tissue may include laser ablations to remove lesions, deep chemical peels, cryotherapy, liposuction, waterjet ablations, and mechanical abrasions to remove or disrupt tattoo inked tissue.

This classification is presented for illustrative purposes to indicate that soft tissue disruption spans a very wide spectrum of modalities, magnitudes, consequences, and medical outcomes. Mechanical disruption of soft tissues spans a wide spectrum of mechanical implements and motions for causing the disruption, a wide spectrum of target tissues amenable to medical or cosmetic treatment, and a wide spectrum of disruption magnitudes that call out for quantification.

Devices for inflicting significant soft tissue disruption may be quantified based on the amount of energy delivered to the tissue. For example, it may be relatively easy to measure the impinging irradiance generated by directed energy sources (medical lasers, focused ultrasound, microwaves, etc.). While the energy actually deposited in tissues or in unwanted foreign matter (like tattoo ink) intimately depends on numerous reflectivities and absorptivities, useful empirical correlations may be made between measured surface irradiance and deposited energy.

Deposited energy density is a primary factor relating to tissue disruption. Disrupting tissues requires breaking structural fibers and other bonds, which requires energy. The amount of structural disruption is proportional to the amount of energy deposited per unit volume of tissue: the energy density.

One class of tissue disruption devices, in which the deposited energy density has not previously been quantified, are those that are fundamentally mechanical in nature: needles, blades, brushes, and all other hard implements designed to mechanically abrade, puncture, cut, rip, tear, or otherwise disrupt and mutilate soft tissue.

### SUMMARY

Embodiments of the present invention may comprise computational tools for the quantification, prediction, simulation, and analysis of soft tissue disruption generated by moving mechanical implements: needles, blades, brushes, and all other hard implements (sharp or otherwise) with the capacity to mechanically abrade, puncture, cut, rip, tear, or otherwise disrupt and mutilate soft tissue. A disrupting implement may be sufficiently strong and hard so that its size and shape are maintained during all interactions with the soft tissue of interest.

A disrupting instrument may comprise various shapes, sizes, and arrangements. The kind of motions employed may comprise various motions, constant or variable, oscillating or not, in one or more directions, and in any combination. The kind of soft tissues acted upon are likewise general. Although skin and dermatological needs may benefit from embodiments of the invention, the tools developed may also be applied to any soft tissue: in vivo or ex vivo, human or animal, and in any body location.

In soft tissue disruption, an implement driver ensures specified motions of hard implements through some soft tissue; the motivating forces required to drive the implement through the tissue. The implement driver provides the force or torque as required to execute the specified kinematic motions. Movement of the implement in soft tissue imparts a certain mechanical energy to the tissue in a certain energy density.

Quantification, prediction, and simulation tools for soft tissue disruption consistent with embodiments of the invention are all founded on the development of a novel, readily computable surrogate for the mechanical energy density delivered to tissues. This surrogate is referred to herein as the kinematic displacement density (KDD).

To accurately predict the mechanical energy density delivered to tissues from first principles requires nonlinear time-dependent computations and detailed knowledge of the constitutive equations connecting dynamic and kinematic quantities. For living tissues, these equations are complex and mostly unknown. Indeed, these equations are often nonlinear, viscoelastic, and irreversible. There is a need to predict the energy density delivered by mechanical implements to tissues.

A disrupting implement may be conceptualized as a piston that displaces a volume of tissue as it moves into and through the tissue. The rate of tissue volume displaced (mm³/s) equals the piston area (mm²) times the piston speed (mm/s) in the direction perpendicular to the area. Piston power, or the rate of mechanical energy delivered to the tissue, equals the piston speed times the tissue drag force acting on the piston, or alternatively, it equals the rate of volume displaced times the mean pressure acting on the piston (drag force divided by piston area). Thus, in the absence of constituency information or direct drag force measurements, the rate of mechanical energy delivered to tissue is proportional to the volume rate of tissue displacement.

This rate may be integrated over time to find the total volume of tissue displaced. This quantity may then be normalized by dividing it by the total volume of tissue worked. The result is a conveniently dimensionless quantity (displaced volume/worked volume), descriptively named the kinematic displacement density. KDD is purely kinematic because dynamic forces are ignored. At any particular point, within the worked tissue, KDD is computed from the motions of the geometric solid that models the disrupting implement. Because the geometry of the implement is known and unchanging, and the driven motions are completely specified, KDD is readily computed.

When summing kinematic displacement, only positive displacements add to the KDD. For example, when a tapered needle penetrates tissue it pushes tissue out of the way, which displacement is responsible for structural tissue disruption. When the same needle retracts, the tissue relaxes back to fill in the vacant space left by the retracting needle. This relaxation or negative displacement is not expected to disrupt tissue, and therefore, it is not counted in the KDD.

The described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure.

### BRIEF DESCRIPTION OF THE DRAWINGS

In order that the advantages of the subject matter may be more readily understood, a more particular description of the subject matter briefly described above will be rendered by reference to specific embodiments that are illustrated in the appended drawings. Understanding that these drawings depict only typical embodiments of the subject matter and are not therefore to be considered to be limiting of its scope, the subject matter will be described and explained with additional specificity and detail through the use of the drawings.

FIG. 1 is diagrammatic representation of an embodiment of the lateral distribution of KDD over a treatment disk;

FIG. 2 is a graph depicting depth distribution of KDD over a treatment disk of an embodiment;

FIG. 3 is a graphical depiction of the shape of an embodiment of a disruption implement, namely a tapering axisymmetric needle; and

FIG. 4 is a graph depicting the disruption implement piston size of the disruption implement embodiment illustrated in FIG. 3.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as Such, multiple figures are presented to separately illustrate the various details of the invention in greater clarity. Similarly, not every embodiment need accomplish all advantages of the present invention.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

It will be readily understood that the components of the embodiments as generally described herein could be arranged and designed in a wide variety of different configurations. Thus, the following more detailed description of various embodiments is not intended to limit the scope of the present disclosure but is merely representative of various embodiments.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Unless otherwise indicated, the terms “first,” “second,” etc. are used herein merely as labels, and are not intended to impose ordinal, positional, or hierarchical requirements on the items to which these terms refer. Moreover, reference to, e.g., a second item does not require or preclude the existence of, e.g., a “first” or lower-numbered item, and/or, e.g., a “third” or higher-numbered item.

Additionally, instances in this specification where one element is “coupled to another element” can include direct and indirect coupling. Direct coupling can be defined as one element coupled to and in some contact with another element. Indirect coupling can be defined as coupling between two elements not in direct contact with each other but having one or more additional elements between the coupled elements. Further, as used herein, securing one element to another element can include direct securing and indirect securing. Additionally, as used herein, “adjacent” does not necessarily denote contact. For example, one element can be adjacent to another element without being in contact with that element.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.”

Unless otherwise indicated, the terms “first,” “second,” etc. are used herein merely as labels, and are not intended to impose ordinal, positional, or hierarchical requirements on the items to which these terms refer. Moreover, reference to, e.g., a second item does not require or preclude the existence of, e.g., a “first” or lower-numbered item, and/or, e.g., a “third” or higher-numbered item.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments are described herein, at least some of the described embodiments allow for the efficient removal of tattoos, permanent makeup, and other indelible mark or pigment on and under the skin, whether they were applied deliberately (as in tattooing) of were acquired naturally (as are pigmented lesions and dermal scarring). While the description herein refers primarily to tattoo removal, the apparatuses, systems, and methods described herein may be also be utilized for any disruption treatments to the skin of a client. Two broad classes of treatments that can be accomplished by the embodiments described herein include superficial dermabrasion (for scar revision and for removal of tattoos, permanent makeup, pigmented lesions, etc.) and axial needling or aesthetic microneedling (for tattooing, skin tightening, wrinkle removal, etc.).

Kinematic displacement density (“KDD”) can be usefully displayed in various formats. For example, KDD may be used, in the form of a kinematic displacement tool (“KD tool”) to better understand the tissue disruption generated in treatments for the removal of tattoo ink or other skin markings. The process may scrub a small circular treatment tile (or tegula) with a vertically oscillating brush of sharp needles to abrade and remove the epidermis and to disrupt the underlying inked dermis. Abrading implements such as the vertically oriented steel needles may simultaneously execute two different motions: a vertical or axial harmonic oscillation (axial refers to the needle axis direction, z), and a horizontal or lateral scrubbing motion that typically follows a spiral path (lateral refers to the directions perpendicular to the needle axis, x and y, which in this case, span the skin surface).

When tissue disruption is sufficient, natural healing processes agglomerate the tissue (once tattoo inked and now disrupted) and expel it from the skin in the form of debris scabs known as eschars. Below the protective scabs the dermis and epidermis are rebuilt, so that when the scabs slough, the new skin in entirely free of tattoo ink. Embodiments of the process and implements for use in tattoo removal or related processes are disclosed, for example, in U.S. Pat. Nos. 8,663,162, 11,020,203, and 11,529,505, and U.S. Patent Application Publication Nos. 2021/0113296 and 2023/0211138, each of which is incorporated herein by reference.

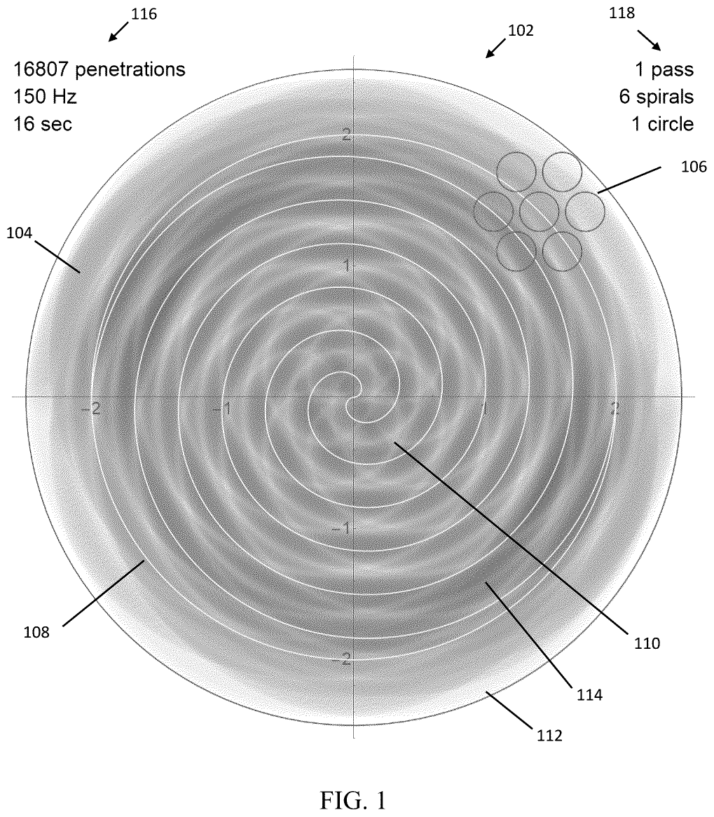

FIG. 1 illustrates an example display of the relative distribution of KDD over a treatment disk 102. KDD in this example is proportional to the density of the background fill 104. The disrupting implement in this example is a six-around-one brush of needles 106, which needles are schematically illustrated and placed for illustration on the outer orbit. The needles 106 are shown in relative size and placement for an embodiment of a disruption brush. The spiral path 108 followed by the brush is overlaid. Because the spiral orbits are relatively dense, KDD is reasonably uniform, except for a network of lessor disruption 110 in the center and a ring of lessor disruption 112 in the far periphery. Variegated annular segments of greater disruption may occur where spiral and circular orbits approach and merge. This illustrative KDD display shown in FIG. 1 covers a single circle using one pass of six spirals. This results in 16807 penetrations at 150 Hz for 16 seconds. Embodiments of the KDD display may include information related to the disruption 116, 118 as illustrated in FIG. 1.

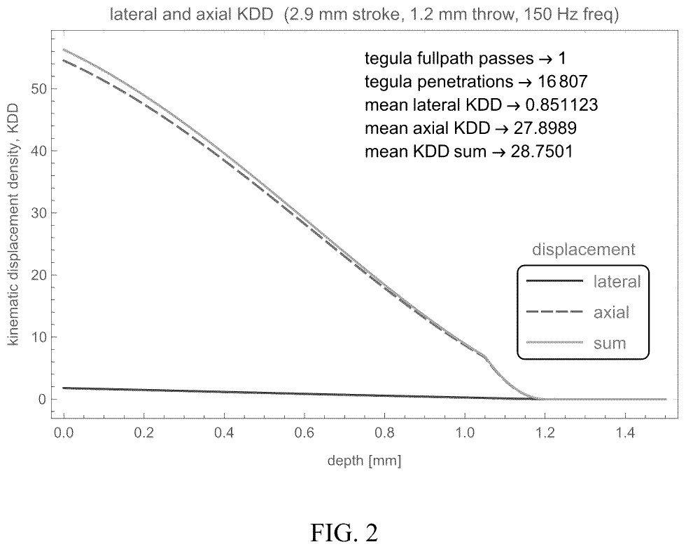

FIG. 2 illustrates an example display of the distribution of KDD with depth. Because the needles execute two motions, there are two KDD components. The “axial” component (dashed line) is the KDD generated by the axially oscillating needles. The “lateral” component (lower solid line) is the KDD generated by the lateral scrubbing movement of the brush. The additive sum of these components is labeled “sum.” Embodiments of KDD display may include information such as the number of passes, number of penetrations, mean lateral KDD, mean axial KDD, mean KDD sum, and other information as would be appropriate or useful for a technician or clinician.

In the embodiment of FIG. 2, the two components of KDD and their sum are plotted. All three monotonically decrease with depth, reaching zero at the maximum set penetration depth of 1.2 mm. Lateral KDD is generated by the lateral translation of the brush. Axial KDD is generated by the axial oscillations of the needles. Axial KDD dominates in this instance because the average needle speed is much greater than the average lateral speed.

With reference to the exemplary displays of FIGS. 1 and 2, the KDD is large, significantly greater than one. Sum KDD equal to 56 means that each volume of tissue at the skin surface was displaced a total of 56 times by the disruption device. In addition, KDD decreases monotonically (almost linearly) with depth, reaching zero at 1.2 mm which was the maximum depth reached by the oscillating needle tips. This is because needle oscillation causes the disrupting implements to spend proportionately more time near the skin surface. In other embodiments of example displays, KDD may approach zero at varying depths according to the maximum depth reached by the disrupting implements.

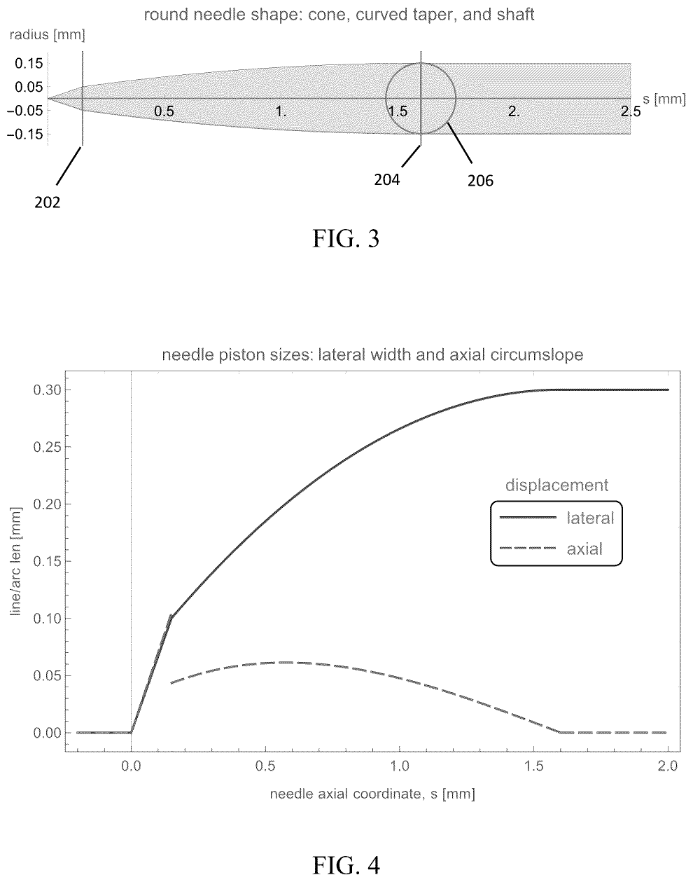

FIG. 3 shows an example display of the implement geometry of the round needle that may be used in conjunction with the KDD display. In this embodiment, the implement geometry is a tapering axisymmetric needle. All needles in the six-around-one brush are identical. Lines mark geometric interfaces: cone-to-taper 202 and taper-to-shaft 204. The circle 206 centered at the taper-to-shaft interface illustrates the size and shape of the constant-diameter shaft.

FIG. 4 illustrates an example display of the piston sizes associated with the disrupting implement illustrated in FIG. 3. In this example, there are two piston sizes, though there may be more or less piston sizes in other displays: lateral width and axial circumslope. Lateral width is associated with lateral motion. The projected differential area perpendicular to lateral motion at any particular needle location (s) equals its width (diameter of a round needle) times a differential depth (Δz).

Axial circumslope is associated with the oscillatory axial motion. The projected differential area perpendicular to axial motion at any particular needle location (s) equals its area derivative (dA/ds) times the differential depth (Δz). For axisymmetric implements like round needles, the area derivative equals the needle circumslope (needle circumference times the radial slope dr/ds). For implements that are not round (like the square or rectangular cross-section teeth associated with electron-beam machined blades), the proper axial size may be given by the more general area relation (dA/ds).

FIG. 4 illustrates that the lateral piston size of a tapered needle increases over the taper length and remains constant along the shaft. In contrast, the axial piston size may increase and decrease over the taper length and is identically zero along the constant-diameter shaft. This means that a constant-diameter shaft generates no axial displacement during penetration, which is the expected result. Only the penetrating tapered portion (the tip cone and curved taper) generates positive axial tissue displacement.

Comparing cumulative piston sizes (the areas under the curves in FIG. 4), in this particular display embodiment, lateral pistons are larger than corresponding axial pistons. Axial cumulative size is limited to the cross-sectional area of the shaft, while lateral cumulative sizes are theoretically indefinite, but of course, practically limited by the depth the target tissue can usefully be penetrated. Although the lateral piston size is everywhere bigger than the axial piston size, axial displacement dominates the KDD in this instance as shown in FIG. 2 because the axial penetrating speed is much larger in this example than the lateral scrubbing speed. It will be appreciated that other cases may vary in relative sizes and displacement ratios and quantities.

Embodiments of a kinematic displacement tool (“KD tool”) may comprise a visual, interface, an underlying KD computational kernel, and hardware components to extend the usage of a KD platform. The KD tool may run on a computer, smart phone, portable tablet, or any other computational platform. For example, some embodiments may comprise applications that communicate with the KD tool as hosted on an Internet server. Some embodiments may facilitate collecting fees and distributing advertisements (for industry disruption devices and special implements, and for KD tool add-ons). The KD tool may comprise applications that enable modification, updating, and security of the KD tool.

The KD tool may have various usage applications. Embodiments of the KD tool may be used in a variety of categories, including, without limitation: (1) simulation/analysis of disruption procedures; (2) training of disruption technicians; (3) disruption device design and optimization; (4) programming of semi-automatic disruption devices; and (5) quantitative analysis of mechanical energy density. Paragraphs to follow supply details for each category. In the following, technician refers broadly to the person doing the tissue disruption, while clinician refers to the supervising cosmetic/medical authority. It will be appreciated that the two roles can be carried out by the same person or multiple persons.

A disruption procedure is any cosmetic or medical procedure that uses soft tissue disruption as a means of treatment. Tissue disruption may be accompanied by solutions or ointments that facilitate the disruption, some form of diagnosis or pretreatment, an aftercare regime, and follow-up treatments and exams. KDD simulations may allow the technician/clinician to plan a novel procedure or to understand the outcome of a novel procedure that did not work as expected. Because biological tissues are involved, cosmetic/medical outcomes may be a complex interplay between technician actions and biological responses. KDD maps may provide useful quantitative insight about a specific type or instance of tissue disruption that otherwise would be unavailable to the informed clinician.

During tattoo removal treatments, the disruption technician may scrub the oscillating brush over a small area in a complex pattern. This pattern may be a uniformly spaced spiral with constant lateral speed that requires variable orbit times-orbital times increase with the orbit radius. This may be done while keeping the skin properly stretched, the brush vertically oriented, and watching for liquid overflow of the facilitating solution. It may be a difficult task to do well and especially to learn, because little or no feedback is available. With practice, the action can become muscle memory, but it may be advantageous to have feedback to indicate if the procedure is being done correctly.

In some embodiments, the KD tool may be connected to a precision track pad upon which the disruption device rides; a blunt training tip may replace the sharp needles. The KD tool records the (x, y) lateral positions versus time as the prospective technician practices their technique on the track pad. The KD tool may generate and displays a KDD versus XY plot based on the recorded path and the disruption device parameters: needle geometry and penetration depth, brush layout, and oscillation frequency and stroke (peak-to-peak amplitude). This provides immediate feedback to the technician. The technician trains by modifying their path position and speed to make the KDD plot look as uniform as possible and to reduce the KDD standard deviation over the treated area. In addition, an experienced technician may want to quantitatively check their proficiency from time to time. This may be facilitated with a stand-alone KD computational unit that includes a built-in precision track pad.

Embodiments of the KD tool may also be used to optimize the implement positions for a semi-automatic disruption device that utilizes machine rotation rather than manual path tracking. The rotating implement holder, which may be a single-use cartridge or other structural form, is referred to as the rotor. A semi-automatic disruption device may speed the procedure and relieve the technician burden. In embodiments of a semi-automated disruption device, path flexibility of a manually operated device may be traded for the precision, repeatability, and speed of a semi-automated device. Embodiments of the KD tool may be used to optimize placement of a set number of disruption implements in a rotor to achieve improved uniformity of soft tissue disruption.

In such embodiments, the user chooses the starting locations of the tissue disrupting implements (needles, blades, etc.) and an optimizable merit function: for increased KDD uniformity, the computed standard deviation may be minimized; for increased minimum spot KDD, the least computed spot value (averaged over a small disk) may be maximized; for the lowest maximum KDD, the greatest computed spot value may be maximized; and the like as would be understood by one of ordinary skill in the art. The KD tool may then search the multi-dimensional state space that defines all movable implement positions. This may be done with a simplex or other multi-dimensional optimization algorithm. After state-space convergence, the optimum implement locations may be displayed.

In further embodiments, the KD tool may be used in conjunction with programming semi-automatic disruption devices. When dealing with semi-automatic disruption devices having multiple degrees of freedom, device program development and coding may become a significant obstacle for use. As an example, assume a system with four different mechanical motions: oscillation frequency; penetration depth, speed, ramp, etc.; rotation direction, speed, ramp, etc.; and pumping volume flow rate (to supply a facilitating solution). The system can execute all four motions independently or collectively in near-infinite combinations. Embodiments of the KD tool may provide for this system or any other multi-motion device to be easily and conveniently programmed by the clinician/technician to achieve their tissue disruption goal.

It may not be possible to optimize the disruption program because the solution depends fundamentally on unknown dynamical properties of the tissue. However, an experienced clinician may, for example, want to rotate quickly with shallow non-oscillating implements, then penetrate deeper in several steps using oscillations, slow back-and-forth rotations, and pulses of the facilitating solution. The KD tool may provide for semi-automatic device programming and may visually lay out multiple timeline tracks similar to those used in video and audio editing. The KD tool may comprise one or more tracks for each fundamental motion: oscillation, depth, rotation, and pumping. Multiple tracks may be needed for complicated motions. For example, rotation and depth controls both involve positions, directions, speeds, and speed changes (steps, ramps, etc.) that may be more visually understandable with two separate tracks: one for position and another for speed. Placing the multiple timeline cursor at some point on the timeline will show the device state and cumulative KDD at that time. Dragging the cursor along the timeline will show the changing device state and changing KDD.

With embodiments of the KD tool, the technician/clinician may modify steady state conditions (rotation speed, oscillation frequency, implement depth, pumping flow rate, etc.) on the timelines, as well as add, modify, or delete any number of transitions between steady states or next to other transitions. In this way, the program that operates the semi-automatic disruption device can be constructed piece-by-piece while visually verifying the program along the way. This programming tool may accomplish four tasks, among others: (1) construct the program and display the operation of a selected semi-automatic disruption device, visually illustrating its states and actions; (2) compute and display the predicted KDD versus time; (3) generate the downloadable code necessary to run the device; and (4) directly run the semi-automatic device (if it is available, connected, and appropriately configured).

In further embodiments, the KD tool may be used for quantitative analysis of mechanical energy density. One of the many benefits of KDD may be that it is a computable kinematic surrogate of the dynamical quantity actually proportional to soft tissue disruption, which is the mechanical energy density (“MED”) delivered to the soft tissues. The KD tool may extend its kinematic computations to include dynamics, and MED in particular, when the drag forces on the penetrating and moving implements are directly measured during the tissue disruption procedure.

Measuring implement drag in real time would involve integrating force transducers of one form or another into the disruption device, which may be simple or very difficult depending on the device and closeness of the measurement to the tissue disrupting implement. A simple method measures the torque of the driving motors, or the current and power drawn by the motors, but this may only yield useful implement-referenced data for direct and efficient mechanical transmissions. For other disrupting implements, small torque and force sensors (strain gauges, solid state sensors, and various micro-electro-mechanical sensors) may effectively monitor drag at the brush or rotor level. Measuring implement drag directly may be much more difficult and costly, and the numerous implement data resulting may not be that much more useful than the integrated brush or rotor data. Accordingly, drag data may be used as it becomes available.

KDD's connection to MED may change with time as soft tissue is disrupted. The connection function in some circumstances is expected to decrease exponentially with time as the same volume of tissue is reworked. Depending on the half-life of the connection function, the decay may be slight, as a linear fall, or it may dramatic, as a rapid drop to some small value close to zero connection. Zero connection would mean that the input KDD would produce no resultant MED, which could only occur if the implement drag forces fell to zero.

If the ongoing mechanical disruption continually disrupts never-disrupted, virgin tissue, the connection from KDD to MED would remain constant, assuming the mechanical properties of the tissue remain constant and the integrity of the disruption implement remains constant (that is, it does not dull nor get clogged with debris). However, as the same volume of tissue is reworked, its tissue is disrupted, which reduces its structural integrity, which in turn decreases the implement drag forces, and hence, the mechanical energy density delivered to the tissues. Because MED should proportionately destroy tissue structure and hence proportionately reduce implement drag, its decay should be exponential, dropping not to zero but to some value more indicative of a viscous fluid drag.

Monitoring the decay of tissue MED for a specified KDD gives information about the state of tissue disruption and even the mechanical properties of the disrupted tissue (the weaker the tissue, the quicker it fails). It may also provide a procedural endpoint based on real time measurements that quantify the remaining soft tissue structure.

Embodiments of the KD tool may comprise various components including software components and hardware extensions. Software components may comprise the following elements:

A database describing, among other things, commercially available disruption devices, motion parameters (speed, frequency, depth, etc.), brush and rotor layouts, geometries of individual disruption implements (needles, blades, etc.), geometries of mathematically flexible components (cone, pyramid, frustum, curved tapers, shafts with round, square, or rectangular cross sections, etc.) for constructing novel implement geometries, and geometries of mathematically flexible arrays for defining novel brushes, blades, and rotors.

Selection interfaces and methods for choosing and defining the mechanical disruption device, its implements and motion parameters. Visual drag and drop from pallets, menu selection, and parameter input may be utilized and integrated.

Constructors for assembling selected, mathematically flexible, components into implements and assemblies, which are checked/restricted to be usable from both physical and tissue disruption viewpoints. For example, many novel round needle implements can be constructed from a tip cone, tapered section (straight or curved), and shaft. An acceptable implement may be flexibly general with certain necessary restrictions. For example, the cross-sectional diameter may a continuous and monotonic non-decreasing function of distance from the needle tip.

A multiple-timeline interface for multi-motion programming, which program is checked/restricted to be physically achievable. Each motion may be defined by one or more timelines. Timelines for the same motion may be analytic derivatives or integrals of each other (for example, both a position and speed timelines may be useful for depth and rotation). The interface may be configured such that when one timeline is changed, other derivative or integral timelines are automatically updated; or if an impossibility is detected, the change is properly restricted (for example, steps in depth or rotation angle are physically impossible). Each timeline may consist of any number of steady-state intervals and transitions (step, ramp, or push). Steps define discontinuous transitions; ramps define transitions linear in time; pushes (intimating smooth accelerations driven by forces) define transitions quadratic in time. In general, transitions of positive degree n, which are described by degree-n polynomials in time, are derivatives of transitions of degree n+1. Thus, ramps and pushes are transitions of degree 1 and 2, respectively.

In embodiments of the KD tool, the value associated with each steady-state interval is input and displayed above the horizontal line representing the steady-state motion; to save vertical space (so all timelines can be comprehended together), all steady-state motion lines can be placed at the same height on the timeline (in other words, height gives no information about the actual steady-state value, which is why its numerical value is displayed). In this vertically compressed view, transitions are displayed as vertical start and stop lines (called time dividers) enclosing one or more stacked-slash transition symbols and, when appropriate, a numerical transition rate (velocity, acceleration, etc.). The stacked-slash symbol (a stack of n short, inclined lines) visually indicates the transition direction (up or down) and its polynomial degree. Time dividers have handles. When dragging any time divider, it can exclusively shrink and expand the adjacent intervals (when only that one divider is moved), or alternately, it can also push or pull all time dividers on either its positive (right) or negative (left) time side; all affected transition polynomials are automatically recomputed. A transition can be incrementally added as a null time divider that breaks one steady-state interval into two intervals. When either adjacent interval value is changed the null turns into a step transition, which is indicated by the time divider acquiring an up or down pointing arrowhead (the step symbol). Clicking the step symbol allows the user to broaden and soften the step transition by selecting a ramp or push transition.

Software components may also comprise a graphical-history interface for multi-motion programming, which acts as a visual supplement to the multiple-timeline interface. Vertically compressed and stacked timelines may provide for quick, efficient, and comprehensible multi track programming by collapsing transition information into symbols (the step and stacked-slash symbols). These symbols may hide the computed polynomials that define the motion program for the semi-automatic device. A larger graphical-history interface may display (without vertical compression) the actual piecewise polynomial histories that define the multi-motion program. One history (which is some function of time) may be plotted for each timeline. Color coding may visually connect timelines and their plotted histories. Two dimensional handles can be added to the piecewise interfaces associated with the one-dimensional time-divider handles. Dragging these 2-D handles, simultaneously moves the associated time divider (by the horizontal drag) and modifies the transition polynomials (by the vertical drag). All affected histories and timelines may be recomputed, and displays are updated.

A program parameter panel for multi-motion programming may be used. One of the problems encountered using a graphical interface, where clicks and drags are used to define values, is that the actual values input are often hidden or inaccessible. The program parameter panel allows the user to see and directly modify the parameters that define the polynomial transitions. All affected transition polynomials are automatically recomputed, and all displays are updated.

Software components may also comprise computational algorithms for KDD, MED, etc. and optimization algorithms for rotor and blade design.

Hardware components for embodiments of the KD tool may comprises: precision track pad and interface for manual device training; device digital interfaces to connect a semi-automatic disruption device and other necessary auxiliary devices (for example, a pump to supply a facilitating fluid) for operation or program download; drag force sensors to measure (or infer) implement drag forces during tissue disruption; and sensor analog interfaces for reading and recording real-time drag-force data.

Embodiments of a KD tool consistent with the disclosure herein may comprise: methods of using a computable surrogate for the mechanical energy density delivered to soft tissues by mechanical disruption devices and implements, such as a kinematic displacement density, to analyze, predict, and simulate the mechanical disruption of said soft tissues; software or other tools using KDD to simulate mechanical disruption of soft tissues, and to analyze mechanical disruption treatments based on the predicted KDD produced; software or other tools using KDD for optimizing placement of disruption implements; software or other tools using multiple timelines and graphical histories for programming semi-automatic multi-motion general purpose devices and systems, disruption devices, and disruption devices producing an acceptable KDD result; integrating sensors with mechanical disruption devices to measure or infer implement drag forces encountered during soft tissue disruption; and software or other tools combining input KDD and resultant drag force measurements to compute the Mechanical Energy Density or MED delivered to soft tissues during disruption, to measure the decay history of tissue disruption, to use the decay history to deduce structural properties about the tissue, and/or to signal in real time the proper endpoint of tissue disruption for a particular procedure encountering different tissues with a range of structural properties.

What is claimed is:

1. A system for providing information relating to mechanical energy imparted to soft tissue in relation to a tissue disruption treatment, the system comprising:
a kinematic disruption tool comprising a display; and
a tissue disruption device connected with the kinematic disruption tool;
wherein the kinematic disruption tool:
calculates a surrogate for the mechanical energy density delivered to the soft tissue by the tissue disruption device based at least in part on information from the tissue disruption device; and
displays information regarding the surrogate on the display.

2. The system of claim 1, wherein the surrogate is a kinematic disruption density.

3. The system of claim 1, wherein the information from the tissue disruption device comprises information relating to drag force applied on the tissue disruption device by the soft tissue.

4. The system of claim 1, wherein the tissue disruption device comprises a brush.

5. The system of claim 4, wherein the brush comprises a plurality of needles.

6. The system of claim 1, wherein the kinematic disruption tool further comprises a database.

7. The system of claim 6, wherein the database comprises information relating to a characteristic of the tissue disruption device.

8. The system of claim 7, wherein the characteristic of the tissue disruption device comprises a geometry of the tissue disruption device.

9. The system of claim 1, wherein the kinematic disruption tool further comprises an interface that receives input from a technician.

10. The system of claim 9, wherein the input from the technician comprises selecting one or more elements of a timeline.

11. The system of claim 10, wherein the one or more elements of a timeline comprise a steady-state interval and a transition.

12. The system of claim 9, wherein the input from the technician comprises selecting the tissue disruption device.

13. A method of providing information relating to mechanical energy imparted to soft tissue in relation to a tissue disruption treatment, the comprising:
calculating a surrogate for the mechanical energy density delivered to the soft tissue by a tissue disruption device based at least in part on information relating to a characteristic of a tissue disruption device and on information relating to movement of the tissue disruption device; and
displaying information regarding the surrogate on a display.

14. The method of claim 13, wherein the information relating to the characteristic of the tissue disruption device comprises information relating to a geometry of the tissue disruption device.

15. The method of claim 13, wherein the information relating to movement of the tissue disruption device comprises information relating to a depth of penetration of the tissue disruption device into the soft tissue.

16. The method of claim 13, wherein the information relating to movement of the tissue disruption device comprises information relating to a frequency of penetration of the tissue disruption device into the soft tissue.

17. The method of claim 13, wherein the information relating to movement of the tissue disruption device comprises information relating to drag force applied on the tissue disruption device by the soft tissue.

18. The method of claim 13, wherein the information regarding the surrogate displayed on the display comprises information related to technician training.

19. The method of claim 13, wherein the information regarding the surrogate displayed on the display comprises information related to disruption device optimization.

20. The method of claim 13, wherein the information regarding the surrogate displayed on the display comprises information related to programming of a semi-automatic disruption device.
